(19) United States
(12) Patent Application Publication
(10) Pub. No.: US 2007/0208407 A1
(43) Pub. Date: Sep. 6, 2007

(54) Medical device delivery systems

(76) Inventors: Michael Gerdts, Big Lake, MN (US); Karen Turner, Lino Lakes, MN (US)

Correspondence Address: FISH & RICHARDSON PC, P.O. BOX 1022, MINNEAPOLIS, MN, 55440-1022, US

(21) Appl. No.: 11/368,544

(22) Filed: Mar. 6, 2006

(51) Int. Cl. A61F 2/06

(52) U.S. Cl. 623/1.11

(57) ABSTRACT

Implantable medical endoprosthesis delivery systems and articles are provided.

### TECHNICAL FIELD

The invention relates to medical device delivery systems, and to related methods and components.

### BACKGROUND

Systems are known for delivering medical devices, such as stents, into a body lumen. Often, such systems include a proximal portion that remains outside the body during use and a distal portion that is disposed within the body during use. The proximal portion typically includes a handle that is held by an operator of the system (e.g., a physician) during use, and the distal portion can include an outer member surrounding an inner member with a stent positioned therebetween. Generally, the operator of the system positions the distal portion within the lumen at a desired location (e.g., so that the stent is adjacent an occlusion). The operator can then retract the outer member to allow the stent to engage the occlusion/lumen wall. Thereafter, the operator removes the distal portion of the system from the lumen.

### SUMMARY

In general, the invention relates to implantable medical endoprosthesis delivery systems (e.g., stent delivery systems), as well as related components and methods. The systems can be used, for example, to deliver a medical endoprosthesis (e.g., a stent) to a desired location within a lumen of a subject (e.g., an artery of a human).

Generally, the systems relate to implantable medical endoprosthesis delivery systems that include an inner member, a retractable outer member, an implantable medical endoprosthesis disposed between the inner and outer members, and optionally a bumper proximal to the implantable medical endoprosthesis. In a delivery configuration, the endoprosthesis is constrained within the outer member in a reduced-diameter configuration. During deployment, the outer member is retracted proximally, releasing the endoprosthesis and allowing the endoprosthesis to expand. The bumper, if present, can reduce the ability of the endoprosthesis to move proximally as the outer member is retracted.

The systems are configured to increase the friction between the implantable medical endoprosthesis and/or the inner member relative to the outer member to an extent that the friction is sufficient to at least partially resist the release of compression forces on the inner member and/or the implantable medical endoprosthesis that might arise from the retraction of the outer member. Generally, the friction force between the implantable medical endoprosthesis and/or the inner member and the outer member remains greater than the compression force at least until such time as the distal-most part of the implantable medical endoprosthesis (the first part of the endoprosthesis to be exposed upon retraction of the outer member) has contacted the walls of the lumen in which it is being deployed. In this fashion, the system reduces, e.g., prevents, the compression forces from being imparted into the endoprosthesis prior to its being partially implanted, at which point the implantation will reduce the likelihood of longitudinal movement of the endoprosthesis. Such may result in greater accuracy of deployment.

Embodiments may include one or more of the following advantages.

In some embodiments, the predictability, accuracy, and/or reproducibility of deployment location of the implantable medical endoprosthesis can be enhanced.

In certain embodiments, the longitudinal displacement of the implantable medical endoprosthesis during deployment can be reduced (e.g., can be eliminated).

Other features and advantages of the invention will be apparent from the description, drawings and claims.

### DESCRIPTION OF DRAWINGS

FIG. 1 is a cross-sectional view of an embodiment of an implantable medical endoprosthesis delivery system.

FIG. 2 is a transverse cross-sectional view, taken along line 2-2, of the embodiment of FIG. 1.

FIG. 3 is a transverse cross-sectional view, taken along line 3-3, of the embodiment of FIG. 1.

FIG. 4 is a cross-sectional view of an embodiment of an implantable medical endoprosthesis delivery system.

FIG. 5 is a transverse cross-sectional view, taken along line 5-5, of the embodiment of FIG. 4.

FIG. 6A is a transverse cross-sectional view of an embodiment of an implantable medical endoprosthesis delivery system.

FIG. 6B is a transverse cross-sectional view of an embodiment of an implantable medical endoprosthesis delivery system.

FIG. 7A is a cross-sectional view of an embodiment of an implantable medical endoprosthesis delivery system.

FIG. 7B is a cross-sectional view of the embodiment of FIG. 7A in which the implantable medical endoprosthesis is in a partially-deployed state.

FIG. 8 is a cross-sectional view of an embodiment of an implantable medical endoprosthesis delivery system.

FIG. 9 is a transverse cross-sectional view, taken along line 9-9, of the embodiment of FIG. 8.

FIG. 10 is a cross-sectional view of an embodiment of an implantable medical endoprosthesis delivery system.

FIG. 11A is a transverse cross-sectional view of an embodiment of an implantable medical endoprosthesis delivery system.

FIG. 11B is a transverse cross-sectional view of an embodiment of an implantable medical endoprosthesis delivery system.

FIG. 11C is a transverse cross-sectional view of an embodiment of an implantable medical endoprosthesis delivery system.

FIG. 11D is a transverse cross-sectional view of an embodiment of an implantable medical endoprosthesis delivery system.

FIG. 11E is a transverse cross-sectional view of an embodiment of an implantable medical endoprosthesis delivery system.

FIG. 12 is a partial cross-sectional view of an embodiment of an implantable medical endoprosthesis delivery system.

FIG. 13A is a cross-sectional view of an embodiment of an implantable medical endoprosthesis delivery system.

FIG. 13B is a cross-sectional view of the embodiment of FIG. 13A in which the implantable medical endoprosthesis is in a partially-deployed state.

FIG. 14 is a cross-sectional view of an embodiment of an implantable medical endoprosthesis delivery system.

FIG. 15 is a transverse cross-sectional view of an embodiment of an implantable medical endoprosthesis delivery system.

FIG. 16 is a transverse cross-sectional view of an embodiment of an implantable medical endoprosthesis delivery system.

FIG. 17A is a partial cross-sectional view of an embodiment of an implantable medical endoprosthesis delivery system.

FIG. 17B is a cross-sectional view of the embodiment of FIG. 17A in which the implantable medical endoprosthesis is in a partially-deployed state.

Like reference symbols in the various drawings indicate like elements.

### DETAILED DESCRIPTION

Generally, implantable medical endoprosthesis delivery systems are provided that include an inner member, a retractable outer member, an implantable medical endoprosthesis disposed between the inner and outer members. In a delivery configuration, the endoprosthesis is constrained within the outer member in a reduced-diameter configuration. During deployment, the outer member is retracted proximally, releasing the endoprosthesis and allowing the endoprosthesis to expand. The systems are configured to increase the friction between the implantable medical endoprosthesis and/or the inner member relative to the outer member to an extent that the friction is sufficient to at least partially resist the release of compression forces on the inner member and/or the implantable medical endoprosthesis that might arise from the retraction of the outer member. This can be accomplished, for example, either by increasing the coefficient of friction of the outermost surface of the implantable medical endoprosthesis, and/or by configuring the inner and/or outer members to have at least two different portions that have different coefficients of friction. The latter of these can be accomplished, for example, by treating (e.g., coating, roughening, or texturing) part or all of a surface of the inner and/or outer member to create at least two portions different coefficient of friction; forming the inner and/or outer members into at least two portions having different coefficients of friction (e.g., by forming the portions of different materials that have different coefficients of friction); or by adding a wedge or bumper to the inner and/or outer members that is configured to have a different coefficient of friction than the remainder of the inner and/or outer member.

Generally, the friction force between the implantable medical endoprosthesis and/or the inner member and the outer member remains greater than the compression force at least until such time as the distal-most part of the implantable medical endoprosthesis (the first part of the endoprosthesis to be exposed upon retraction of the outer member) has contacted the walls of the lumen in which it is being deployed. In this fashion, the system reduces, e.g., prevents, the compression forces from being imparted into the endoprosthesis prior to its being partially implanted, at which point the implantation will resist reduce the likelihood of longitudinal movement of the endoprosthesis. Such may result in greater accuracy of deployment.

Outer Member with Treated Interior Surface

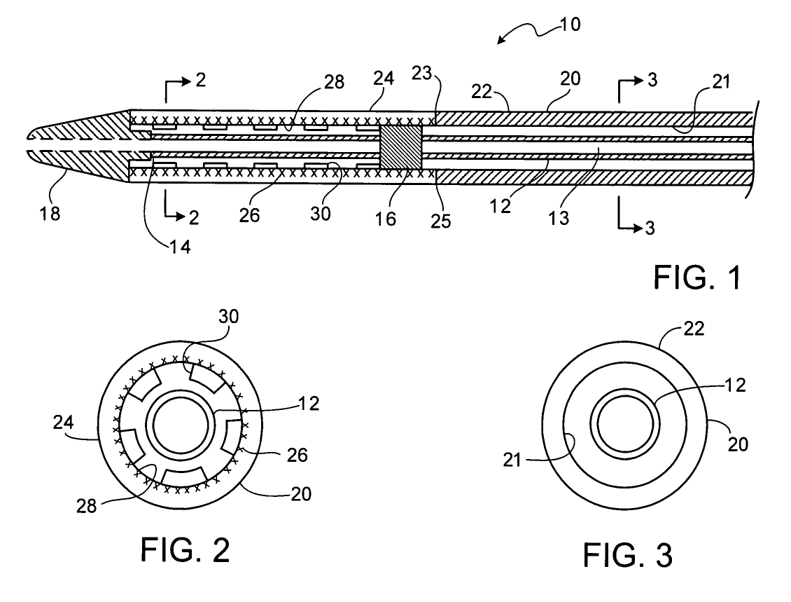

In certain embodiments, for example, as illustrated in FIGS. 1-3, an endoprosthesis delivery device 10 includes an inner member 12 having a lumen 13 (e.g., a guidewire lumen) extending longitudinally therethrough. A distal tip 18 (e.g., a conical or bullet-shaped tip) is attached to the inner member 12 at a distal end 14 of the inner member 12, and a bumper 16 is optionally located proximal to the distal end 14 of the inner member 12. An outer member 20 is disposed about the inner member 12. A self-expanding stent 30 is disposed between the inner member 12 and the outer member 20 such that it extends longitudinally between the conical tip 18 and the bumper 16. Outer member 20 has a proximal region 22 having a distal end 23, and a distal region 24 that extends distally from the distal end 23 of the proximal portion 22. The distal region 24 has a proximal end 25 that is proximal the stent 30 and the bumper 16, such that the distal region 24 of the outer member 20 extends over the stent 30 and the bumper 16.

The distal region 24 of the outer member 20 has an interior surface 28 that is treated (represented by x-marks 26) (e.g., roughened) to have a high coefficient of friction relative to an interior surface 21 of the proximal region 22 of the outer member 20. As referred to herein, the coefficient of friction of a material is measured according to ASTM D1894-01. In some embodiments, the interior surface 28 is treated by roughening the interior surface 28. Processes for roughening a surface include, for example, abrading, etching, scratching, embossing, stamping, melting, and pressing. Also encompassed are methods of molding an article such that the surface is formed with a texture. Roughening can increase the friction between the interior surface 28 of the outer member 20 and the stent 30 when the outer member 20 is retracted. The roughening of the interior surface 28 can be accomplished mechanically, e.g., by abrading the interior surface, chemically, e.g., by etching the interior surface, and/or by ablation (e.g., laser ablation), and/or can be molded directly into the distal region upon formation of the outer member. Exemplary mechanical roughening methods include inserting a mandrel having a textured, roughened or abrasive surface into the distal region of the outer member to abrade the interior surface or otherwise change the interior surface; cutting threads into the interior surface by screwing a thread-cutting mandrel into the distal region of the member; inserting a mandrel having a roughened configuration into the distal region, heating the distal region to a softening point of the material, and compressing the distal region material around the mandrel to impart the roughened configuration into the interior surface of the distal region; utilizing a wire brush to roughen the interior surface; or using a braided or otherwise textured mandrel to impart a texture to the interior surface (e.g., with the aid of heat and/or pressure). Exemplary chemical roughening methods include etching. Etching can include liquid phase etching, e.g., using chromic acid and/or Fluoro Etch (2-methoxyethyl ether 80%, sodium naphthalene 20%), or gas phase etching, such as plasma etching with, e.g., hydrogen, oxygen, and/or argon. Other methods include corona surface treatment of the interior surface.

In some embodiments, the interior surface 28 of the distal region 24 is treated after having been formed into a tube, e.g., after the outer member 20 has been formed. In certain embodiments, roughening is done prior to forming the outer member 20. For example, a sheet of material can have a surface thereof treated to roughen the surface, and the sheet can then be formed into a tube in which the treated surface faces inwardly. Such treatment can include any of those described above. The tube can then be attached to the proximal region 22 of the outer member 20, where the tube becomes the distal region 24 of the outer member 20. In some embodiments, a sheet of material can have a portion of the surface treated to roughen the portion, and a portion left untreated. The sheet can then be formed into a tube in which the treated portion faces the interior, such that the treated portion forms the distal region 24 and the untreated portion forms the proximal region 22 of the outer member 20.

In general, the interior surface 21 of the proximal region 22 of the outer member 20 has a lower coefficient of friction than the interior surface 28 of the distal region 24 of the outer member 20. For example, in certain embodiments, the interior surface 21 of the proximal region 22 has a coefficient of friction that is at least about 10% less (e.g., at least about 20% less, at least about 30% less, at least about 40% less, or at least about 50% less) than the coefficient of friction of the interior surface 28 of the distal region 24 of the outer member 20. In certain embodiments, the interior surface 21 of the proximal region 22 of the outer member 20 is not roughened or otherwise treated to increase friction between it and the stent 30. The lack of treatment facilitates the stent 30 and inner member 12 to be more readily inserted into the outer member 20 and moved to the distal region 24 of the outer member 20. In some embodiments, the interior surface 21 of the proximal region 22 of the outer member 20 is treated to reduce the friction between it and the stent 30. For example, the interior surface 21 can have a lubricious coating, having a lubricious material, applied thereto. Exemplary lubricious materials include PTFE, fluoropolymer, silicone, ultrahigh molecular weight polyethylene, an oil, or blends thereof. Optionally, the lubricious material can be incorporated into the proximal region 22 of the outer member 20.

In certain embodiments, substantially the entirety of the interior surface 28 of the distal region 24 of the outer member 20 is treated. In other embodiments, less than 100% (e.g., less than about 75%, less than about 50%, less than about 33%, less than about 25%, or less than about 20%) of the interior surface 28 of the distal region 24 of the outer member 20 is treated.

In some embodiments, the interior surface 28 of the distal region 24 of the outer member 20 includes a high-friction material in lieu of or in addition to being treated. The high-friction material can provide sufficient friction with the stent 30 to prevent and/or reduce distal movement of the stent 30 upon deployment, optionally without requiring additional treatments, such as roughening of the surface. For example, the distal region 24 of the outer member 20 can be formed of, or have the interior surface 28 lined with, a polymer of tetrafluoroethylene and perfluorovinylether (PFA) rather than the PTFE. Other exemplary high-friction materials include nylon, PEEK, thermoplastic urethane (e.g., Pellathane), and/or polyethylene.

Endoprosthesis with Treated Outer Surface

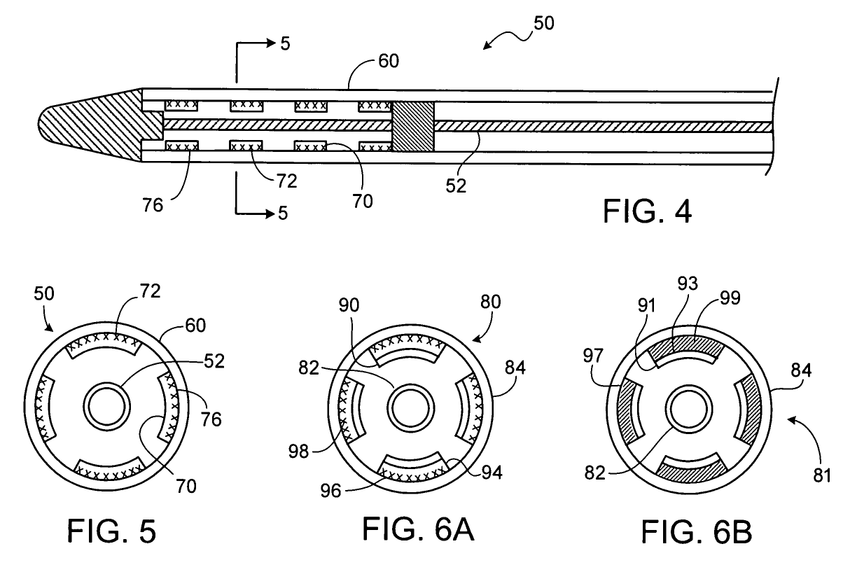

In certain embodiments, for example, as illustrated in FIGS. 4-5, an endoprosthesis delivery device 50 includes an inner member 52 and an outer member 60 concentrically disposed about the inner member 52. A self-expanding stent 70 is disposed between the inner member 52 and the outer member 60. The stent 70 can include a polymer, e.g., a shape-memory polymer, and/or a metal or alloy, e.g., Nitinol, stainless steel, and/or a shape memory alloy. At least a portion of an outer surface 72 of the stent 70 is treated (represented by x-marks 76) (e.g., roughened) to increase the friction between the outer member 60 and the stent 70 when the outer member is retracted.

In some embodiments, the outer surface 72 of the stent 70 is treated by roughening the outer surface 72. The roughening of the outer surface 72 can be accomplished mechanically, e.g., by abrading the outer surface, chemically, e.g., by etching the outer surface, by modifying the chemical finishing process in making the stent, and/or can be molded directly into the outer surface upon formation of the stent. Exemplary mechanical roughening methods include abrading the outer surface, e.g., with a rasp or a wire brush; cutting channels into the outer surface; heating the stent to a softening point of the material making up the outer surface of the stent and molding a roughened pattern into the outer surface material; and/or leaving the outer surface of the stent unpolished such that it retains a roughened surface. Exemplary chemical roughening methods include any of the chemical roughening techniques described above, e.g., etching and/or ablation.

In some embodiment, as illustrated in FIG. 6A, an endoprosthesis delivery device 80 includes an inner member 82, an outer member 84 concentrically disposed about the inner member 82, and a self-expanding stent 90 disposed between the inner member 82 and the outer member 84. The stent 90 has a coating 94 on at least a portion of an outer surface 92 thereof. An outer surface 96 of the coating 94 is treated (represented by x-marks 98) (e.g., roughened) to increase the friction between the outer member 80 and the stent 90 when the outer member 80 is retracted. The outer surface 96 of the coating 94 can be treated by any of the methods described above. The coating 94 can be any material that is biocompatible and that will provide the necessary friction when the outer surface is treated. Exemplary coating materials include etched PTFE (ePTFE) and or yarns. The coating can be applied such that it forms an irregular surface (e.g., the coating can be in braided or woven form). In some embodiments, the coating 94 can be biodegradable.

Generally, in certain embodiments, substantially the entirety of the outer surface of the stent 90 and/or the outer surface 96 of the coating 94 is treated. In other embodiments, less than 100% (e.g., less than about 75%, less than about 50%, less than about 33%, less than about 25%, or less than about 20%) of the outer surface of the stent 90 and/or the outer surface 96 of the coating 94 is treated.

In some embodiments, for example as illustrated in FIG. 6B, an endoprosthesis delivery device 81 includes an inner member 82, an outer member 84 concentrically disposed about the inner member 82, and a self-expanding stent 91 disposed between the inner member 82 and the outer member 84. The stent 91 has a coating 99 on at least a portion of an outer surface 93 thereof. Coating 99 comprises a material having a high enough coefficient of friction to reduce distal movement of the stent 91 upon deployment. The coefficient of friction required to so reduce distal movement will vary, depending on the coefficient of friction of the opposing surface with which the coating 99 is in contact. Exemplary materials of which the outer surface 93 of the stent 91 can be formed or lined with include PFA, nylon, PEEK, thermoplastic urethane (e.g., Pellathane), and/or polyethylene. In certain embodiments, the outer surface 93 of the stent 91 can have a coefficient of friction of at least about 0.15 (e.g., at least about 0.20, at least about 0.25, at least about 0.30, at least about 0.35, or at least about 0.40). Generally, the shorter the stent, the higher the coefficient of friction of the outer surface of the stent. Optionally, the coating 99 can also be treated to roughen the outer surface 97 thereof, which can increase the coefficient of friction of the outer surface 97 of the stent 91.

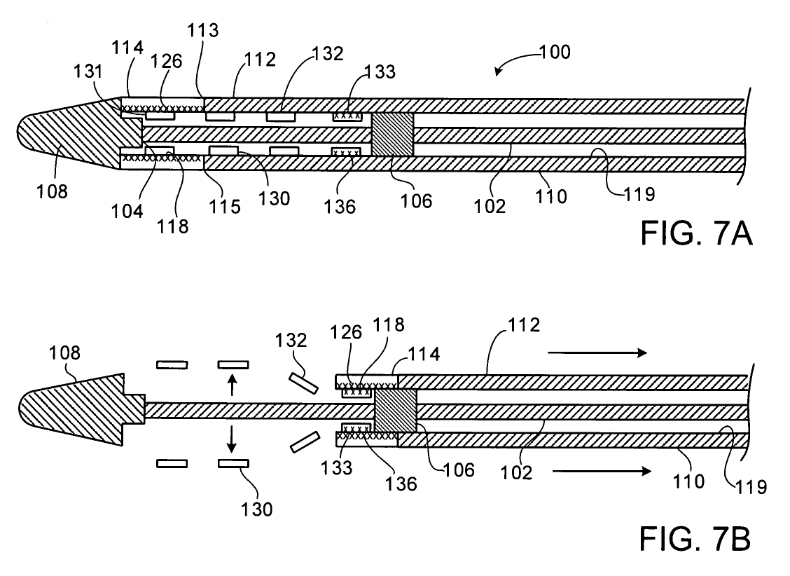

In some embodiments, the system is configured to have the friction increased only as the outer member is partially retracted. This can, for example, allow the endoprosthesis and/or inner member to be restrained from moving distally only as the endoprosthesis is partially deployed. Such a configuration may reduce compressive forces imparted on the endoprosthesis during retraction of the outer member while providing the necessary friction to resist any compressive force that is otherwise imparted on the system. For example, as illustrated in FIGS. 7A (showing the delivery device in a delivery configuration) and 7B (showing the delivery device in a partially-deployed configuration), an endoprosthesis delivery device 100 includes an inner member 102 having a distal tip 108 at a distal end 104 and a bumper 106 located proximal to the distal end 104 of the inner member 102. An outer member 110 is concentrically disposed about the inner member 102. A self-expanding stent 130 is disposed between the inner member 102 and the outer member 110 such that it extends longitudinally between the distal tip 108 and the bumper 106. The outer member has a proximal region 112 and a distal region 114 that extends distally from a distal end 113 of the proximal portion 112. A proximal end 115 of the distal region 114 is proximal a distal end 131 of the stent 120 and distal the bumper 106.

The distal region 114 of the outer member 110 has an interior surface 118 that is treated (represented by x-marks 126) relative to an interior surface 119 of the proximal region 112 of the outer member 110. A proximal region 133 of an outer surface 132 of the stent 130 is also treated (represented by x-marks 136). With this configuration, the treated portions 136, 126, respectively, of the stent 130 and the outer member 110 can increase the friction between the two components (relative to the friction that would exist between the two absent any roughening) as the outer member 110 is retracted. Additionally, when the treated portion 126 of the inner surface 118 of the distal region 114 of the outer member 110 overlays the treated portion 136 of the outer surface 132 of the stent 130, the friction between the two can increase yet again. An increase in friction can reduce the ability of the stent to move distally even as the surface area of contact between the outer member 110 and the stent 130 decreases, which can increase the deployment accuracy of the stent 130. While the illustrated embodiment shows treatment (e.g., roughening) of portions of both the inner surface 118 of the distal region 114 of the outer member 110 and the outer surface 132 of the stent 130, such an effect can also be produced by treating or otherwise increasing the friction of just one of the inner surface 118 of the distal region 114 of the outer member 110 and the outer surface 132 of the stent 130.

High-Friction Wedges

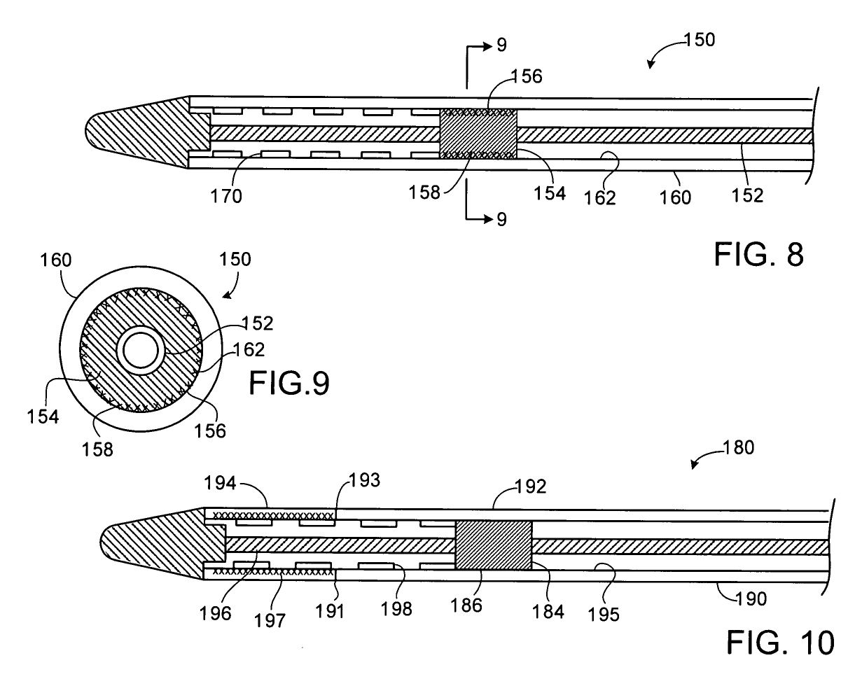

In some embodiments, e.g., as illustrated in FIGS. 8 and 9, an endoprosthesis delivery device 150 includes an inner member 152 and an outer member 160 concentrically disposed about the inner member 152. A self-expanding stent 170 is disposed between the inner member 152 and the outer member 160. A cylindrical wedge 154 having an outer surface 156 is attached to and disposed about the inner member 152 at a location proximal to the stent 170. The wedge 154 has a diameter sufficient for the outer surface 156 to contact an inner surface 162 of the outer member 160. The outer surface 155 of the wedge 154 includes a portion that is treated (represented by x-marks 158) to increase the friction between the outer member 160 and the wedge 154 (and through the wedge, the inner member 152) when the outer member 160 is retracted. Thus, instead of providing increased friction between the stent 170 and the outer member 160 to reduce the ability of the inner member 152 from moving distally and propelling the stent 170 in a distal direction, system 150 relies on friction between the wedge 154 and the outer member 160 to reduce the ability of the inner member 152 from moving distally.

In some embodiments, the outer member has an inner surface that is treated (e.g., roughened, etched, and/or formed of and/or coated with a tacky and/or high friction material) to increase the friction between the treated portion and the outer surface of the wedge. For example, FIG. 10 illustrates an endoprosthesis delivery device 180 that includes an inner member 182 and an outer member 190 concentrically disposed about the inner member 182. A self-expanding stent 198 is disposed between the inner member 182 and the outer member 190. A cylindrical wedge 184 having an outer surface 186 is attached to and disposed about the inner member 182 at a location proximal to the stent 198. The outer member has a proximal portion 192 and a distal portion 194 that has a proximal end 193 connected to a distal end 191 of the proximal portion 192. The distal portion 194 of the outer member 190 has an interior surface 196 that is etched (represented by x-marks 197) to increase the friction between the outer member 190 and both the stent 198 and the wedge 184 when the outer member 190 is retracted.

In some embodiments, the wedge 184 can be at least partially formed of or at least partially coated with a high-friction material (e.g., PFA, nylon, PEEK, thermoplastic urethane (e.g., Pellathane), and/or polyethylene). In other embodiments, the wedge can be at least partially formed of or at least partially coated with a tacky material (e.g., a polyether-type thermoplastic polyurethane (PTU) such as, for example, a polymer from the Tecothane® family of polymers). The high-friction or tacky material is selected to have a sufficiently high coefficient of friction to provide sufficient friction, for a given surface area of contact with the inner surface 196 of the distal portion 194 of the outer member 190 to reduce (e.g., prohibit) distal movement of the stent 170 upon deployment.

In certain embodiments, the wedge is configured to have an outer surface having a coefficient of friction of at least about 0.15 (e.g., at least about 0.20, at least about 0.25, at least about 0.30, at least about 0.35, or at least about 0.40).

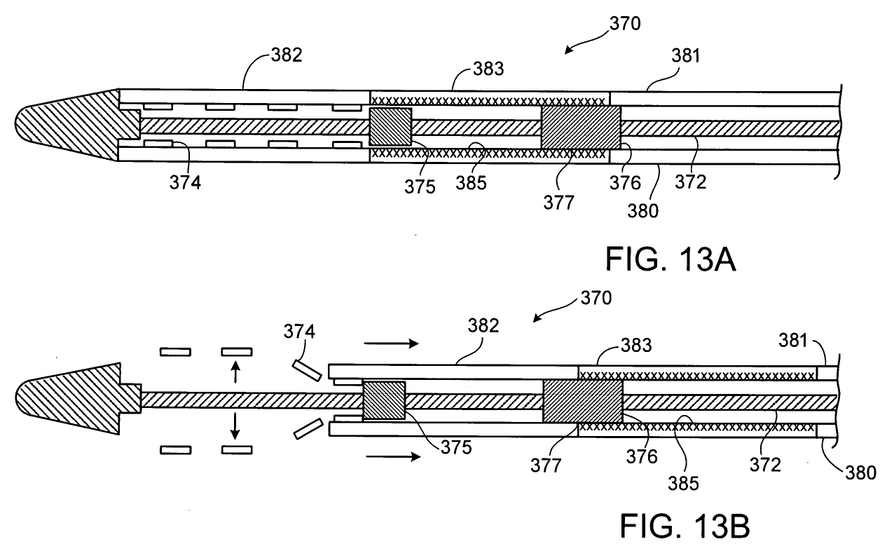

In some embodiments, for example, those illustrated in FIGS. 8-10, the wedge can function as a bumper, e.g., can be located just proximal to the endoprosthesis to reduce proximal movement of the endoprosthesis as the outer member is retracted. In other embodiments, for example, as illustrated in FIGS. 13A and 13B (discussed in detail below) the wedge can be a separate element form a bumper. Generally, a bumper is located just proximal to the pre-deployed endoprosthesis, and need only have a diameter large enough to ensure that the distal edge of the bumper can contact the proximal edge of the endoprosthesis and reduce the ability of the endoprosthesis to move proximally. For example, a bumper that is attached to the inner member need not be large enough in diameter to contact the outer member, so long as it is large enough in diameter to contact the proximal edge of the stent. A wedge, on the other hand, generally contacts both the inner member and the outer member to cause friction to arise between the wedge and the inner and/or outer member upon retraction of the outer member.

In certain embodiments employing a wedge, even when the endoprosthesis is close to fully deployed, the wedge can reduce distal movement of the inner member by providing friction between the inner member and the outer member. Thus, where the endoprosthesis is particularly short, such that it is almost fully deployed before it contacts the lumen walls, the configuration of the delivery system can reduce distal movement. For example, in some embodiments in which a wedge is employed, the endoprosthesis can be no more than about 60 mm (e.g., no more than about 55 mm, no more than about 50 mm, no more than about 45 mm, no more than about 40, no more than about 35 mm, or no more than about 30 mm) long.

The length of the wedge is selected to provide sufficient friction while keeping the force necessary to effect retraction of the outer member to acceptable levels. Generally, where shorter endoprostheses are utilized (and thus, generally, less friction is generated between the endoprosthesis and the outer member), the wedge is lengthened to compensate. In some embodiments, the wedge is no less than about 2 mm (e.g., no less than about 3 mm, no less than about 4 mm, no less than about 5 mm, no less than about 6 mm, no less than about 7 mm, no less than about 8 mm, or no less than about 9 mm) long and/or no more than about 10 mm (e.g., no more than about 9 mm, no more than about 8 mm, no more than about 7 mm, no more than about 6 mm, no more than about 5 mm, no more than about 4 mm, or no more than about 3 mm) long. The wedge can have a treatment on the outer surface thereof that imparts friction, or can have a coating that is treated (e.g., roughened) to increase friction. The treatment and/or coating can be any of those discussed above with respect to the inner member, outer member and/or stent. The wedge, and/or an optional coating on an outer surface of the wedge, can include a high-friction material in accordance with those disclosed above.

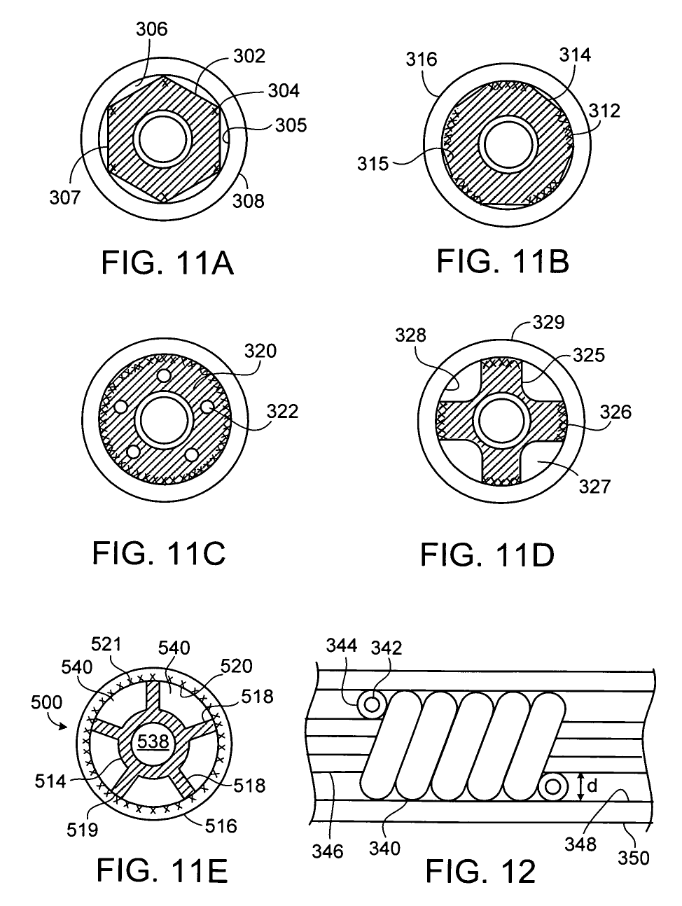

The wedge can be cylindrical, such that substantially the entire outer surface of the wedge contacts the inner surface of the outer member. Alternatively, the wedge can be configured such that a portion of the wedge contacts the inner surface of the outer member while a portion of the outer surface of the wedge does not contact the outer surface of the member. For example, a wedge 302 can have a substantially polygonal shape as in FIG. 11A, with the points 304 of the wedge 302 contacting an inner surface 305 of the outer member 308. Fluid can flow through longitudinal channels 306 between the sides 307 of the wedge 302 and the inner surface 305 of the outer member 308. As another example, a wedge 314 can have a partially polygonal shape having portions 312 contoured to match the curvature of an inner surface 315 of an outer member 316, as illustrated in FIG. 11B. Instead or in addition to the longitudinal channels of the previous examples, a wedge 320 (FIG. 11C) can include longitudinal through-holes 322 to permit fluid flow between a distal side of the wedge and a proximal side of the wedge. A wedge 325 (FIG. 11D) can also assume a non-polygonal shape that includes surfaces 326 that contact an inner surface 328 of an outer member 329 while leaving through-channels 327 to allow fluid flow. Friction between the wedges just discussed and the outer member can be achieved in any of the manners disclosed herein.

In some embodiments, such as illustrated in FIG. 11E, an inner member 514 of implantable medical endoprosthesis delivery system 500 includes a series of splines 518, which are configured to interact with a treated inner surface 520 of an outer member 516. The inner member 514 defines an inner lumen 538 (e.g., a guidewire lumen), while an outer lumen 540 is defined between the inner member 514 and outer member 516. The configuration of the splines 518 allows for contact between the inner member 514 and the outer member 516 while allowing for fluid flow between the splines 518 in the outer lumen 540. While the illustrated embodiment shows the inner surface 520 of the outer member 516 being treated (represented by x-marks 521) to increase friction between it and the splines, in other embodiments the splines 518 (e.g., the outer member-contacting surfaces 519 of the splines 518) can be treated instead of or in addition to the inner surface 520 of the outer member 516. The splines can function in much the same fashion as the wedges described above.

In some embodiments, for example, as illustrated in FIG. 12, wedge 340 can include a wire 342, optionally having a coating 344, wrapped around an inner member 346 and having a total wire diameter d (inclusive of the wire coating 344) of sufficient size that the wire coating 344 contacts an inner surface 348 of an outer member 350. The wire 342, optional wire coating 344, and/or inner surface 348 of the outer member 350 can be treated and/or made of a material or materials to increase the friction between the wedge and the outer member. In some embodiments, the wire 342 and/or the coating 344 is formed of a material having the appropriate flexibility and strength. Examples of materials include metals, alloys and polymeric materials. Examples of metals include platinum, gold and stainless steel. Examples of alloys include gold-containing alloys, platinum-containing alloys, stainless steel and shape memory alloys. Examples of shape memory alloys include Nitinol, silver-cadmium (Ag—Cd), gold-cadmium (Au—Cd), gold-copper-zinc (Au—Cu—Zn), copper-aluminum-nickel (Cu—Al—Ni), copper-gold-zinc (Cu—Au—Zn), copper-zinc/(Cu—Zn), copper-zinc-aluminum (Cu—Zn—Al), copper-zinc-tin (Cu—Zn—Sn), copper-zinc-xenon (Cu—Zn—Xe), iron beryllium (Fe₃Be), iron platinum (Fe₃Pt), indium-thallium (In—Tl), iron-manganese (Fe—Mn), nickel-titanium-vanadium (Ni—Ti—V), iron-nickel-titanium-cobalt (Fe—Ni—Ti—Co) and copper-tin (Cu—Sn). For yet additional shape memory alloys, see, for example, Schetsky, L. McDonald, “Shape Memory Alloys”, Encyclopedia of Chemical Technology (3rd ed.), John Wiley & Sons, 1982, vol. 20. pp. 726-736. Examples of polymeric materials include polyamides (e.g., nylons), thermoplastic polyester elastomers (e.g., Hytrel®), copolyester elastomers (e.g., Arnitel® copolyester elastomers), polyether-block co-polyamide polymers (e.g., PEBAX®) and high-density polyethylene (HDPEs). Coating 344 can be, for example, a polymeric material, such as a plastic (e.g., a thermoplastic) or a thermoset. Examples of polymeric materials include polyamides (e.g., nylons), polyurethanes, styrenic block copolymers, thermoplastic polyester elastomers (e.g., Hytrel®), copolyester elastomers (e.g., Arnitel® copolyester elastomers), polyether-block co-polyamide polymers (e.g., PEBAX®), fluoropolymers (e.g., PTFE, FEP) and HDPEs.

In some embodiments, the point of friction can be set back from the endoprosthesis, such that the endoprosthesis is not subject to higher friction upon retraction of the outer member. An example of such a configuration is illustrated in FIGS. 13A and 13B, in which an endoprosthesis delivery system 370 includes an inner member 372, an outer member 380 concentrically disposed about the inner member 372, and a self-expanding stent 374 disposed between the inner and outer members 372 and 380. The inner member 372 has a bumper 375 located proximal the stent 374, and a wedge 376 located proximal the bumper 375. The outer member 380 includes a proximal region 381, a distal region 382, and an intermediate region 383, configured such that, upon retraction of the outer member 380, the intermediate region 383 will slide over the wedge 376. An inner surface 385 of the intermediate region 383 is treated to increase the friction between the intermediate region 383 and an outer surface 377 of the wedge 376 that contacts the inner surface 385. In some embodiments, the outer surface 377 of the wedge 376 can include a friction-increasing treatment instead of or in addition to the inner surface 385 of the intermediate region 383 of the outer member 380.

This configuration, as can be seen in FIG. 13B, permits deployment of the stent 374 without imparting additional friction between the stent 374 and the outer member 380, because the distal region 382 that overlays the stent 374 is not treated to increase friction. In some embodiments, the distal region 382 can include a treatment designed to decrease friction, e.g., can have a lubricious coating (e.g., a PTFE coating) on an interior surface thereof.

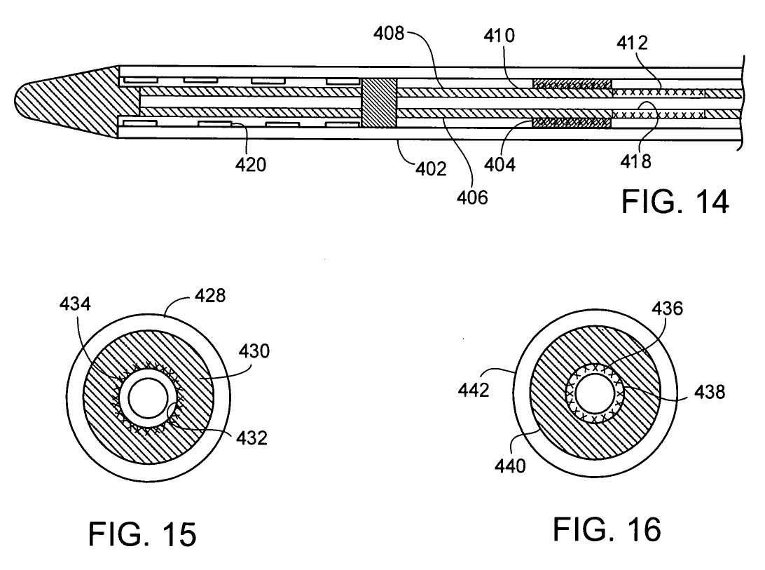

In certain embodiments, the wedge is attached to the outer member and surrounds the inner member, and the friction is generated between the inner member and the wedge upon retraction of the outer member to which the wedge is attached. For example, as illustrated in FIG. 14, a wedge 404 is connected to and disposed within an outer member 402 that is concentrically disposed around an inner member 406. An interior surface 410 of the wedge 404 is configured to surround and contact an outer surface 408 of the inner member 406. The inner member 406 has an intermediate portion 412 that extends proximally from the wedge 404. The wedge 404 is located proximal to a self-expanding stent 420 that is disposed between the inner member 406 and the outer member 402. The interior surface 410 of the wedge and the outer surface 408 of the inner member at the intermediate portion 412 are treated in any of the ways described above to increase the friction between it and the outer surface 408 of the inner member 406. In operation, as the outer member 402 is retracted, the wedge 404 slides proximally over the inner member 406 and the increased friction force between the wedge 404 and the inner member 406 prevents the inner member 406 from moving distally until after the stent 420 is at least partially secured to the walls of the lumen in which it is being deployed. In some embodiments, for example, as illustrated in FIG. 15, only an interior surface 432 of a wedge 430 that is attached to an outer member 428 is treated to increase friction between it and an inner member 434. In other embodiments, for example, as illustrated in FIG. 16, only an outer surface 436 of an inner member 438 is treated to increase friction between it and a wedge 440 that is attached to an outer member 442.

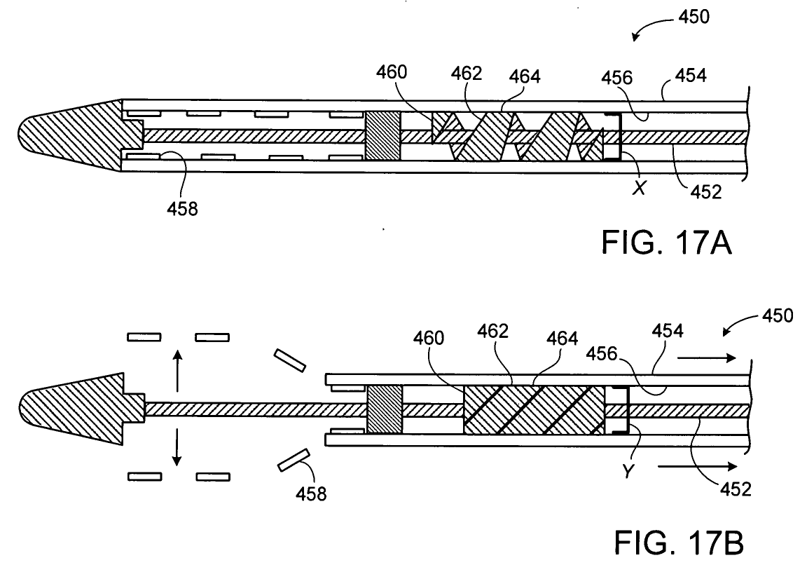

In some embodiments, an example of which is illustrated in FIGS. 17A and 17B, an endoprosthesis delivery system 450 includes a wedge 460, that is formed of a flat wire coil 462, at a location proximal that of a self-expanding stent 458. The coil 462 surrounds and is attached to an inner member 452, and an outer surface 464 of the coil 462 contacts an inner surface 456 of an outer member 454. In a delivery configuration (FIG. 17A), the flat wire coil 462 is in an expanded state, where it will have a first diameter x. The outer surface 464 of the coil 462, the inner surface 456 of the outer member 454, and/or both are selected and/or treated to have an initial degree of friction such that, upon retracting the outer member 454 (as seen in FIG. 17B), the coil 462 is compressed; in other words, the initial degree of friction is sufficient to overcome the resistance to compression of the flat wire coil 462. Upon compressing, the flat wire coil 462 takes on a second diameter y which is at least slightly larger than the first diameter x. This increase in diameter can result in an increase in the friction between the flat wire coil 462 and the outer member 454 to a point sufficient to prevent the inner member 452 from moving distally and causing the stent 458 to move. In addition, the increase in friction can result in an increase in resistance to retraction of the outer member 454 as the stent 458 is deployed, which can provide a tactile signal to the physician that the stent is deployed and implanted to an extent sufficient to anchor the stent in the lumen.

The wedges in certain embodiments are attached to one of the inner and outer members. This attachment can be achieved by adhesive, chemical welding, heat bonding or welding, laser bonding, and/or by mechanical lock. Examples of adhesives include cyanoacrylate adhesives, including medical grade cyanoacrylate adhesives, such as Loctite® brand products available from Henkel Technologies (e.g., Assure™ 425 Surface Curing Threadlocker).

Inner and Outer Member Construction

The inner member and/or outer member can be made of, for example, one or more polymers. Examples of polymers include polyether-block co-polyamide polymers (e.g., PEBAX®), copolyester elastomers (e.g., Arnitel® copolyester elastomers), thermoset polymers, polyolefins (e.g., Marlex® polyethylene, Marlex® polypropylene), high-density polyethylene (HDPE), low-density polyethylene (LDPE), polyamides (e.g., Vestamid®), polyetheretherketones (PEEKs), and silicones. Other examples of polymers include thermoplastic polymers, such as polyamides (e.g., nylon), thermoplastic polyester elastomers (e.g., Hytrel®), and thermoplastic polyurethane elastomers (e.g., Pellethane™). The inner member and the outer member can include the same polymers and/or can include different polymers.

In certain embodiments, the inner member includes a guide wire lumen. In some embodiments, the guide wire lumen can be coated with a polymer (e.g., a polyimide) that can decrease friction between the guide wire lumen and a guide wire that is disposed within guide wire lumen.

In some embodiments, one or more regions of the inner member and/or the outer member can be formed by an extrusion process. In some embodiments, different regions, e.g., different regions made up of different polymers, can be integrally formed. In certain embodiments, different regions can be separately formed and then connected together.

In certain embodiments, the inner member and/or the outer member can be formed of multiple layers. For example, the outer member can include three layers: an outer polymer layer, an inner polymer layer, and an intermediate structural layer disposed between the inner and outer layers. The inner polymer layer can be, for example, polytetrafluoroethylene (PTFE), such as PTFE that has been etched on a surface that is to be bonded to the middle layer (e.g., to improve bonding to other layers). The intermediate structural layer can be, for example, a braid layer. In certain embodiments, the braid layer can be formed of a metal (e.g., tungsten) or metal alloy (e.g., stainless steel). In some embodiments, the braid layer can include one or more flat wires and/or one or more round wires. In certain embodiments, the braid layer can form a pattern between the inner layer and the outer layer. The outer polymer layer can be, for example, nylon, PEBAX®, Arnitel®, or Hytrel®.

In certain embodiments, the outer member and/or the inner member can have one or more translucent regions, or can be formed entirely of translucent material. In some embodiments, the inner member and/or outer member can be formed of multiple polymer layers of differing durometers. In certain embodiments, the inner member and/or the outer member can include multiple coextruded layers. For example, an inner member with an inner layer including HDPE, an outer layer including PEBAX, and a tie layer between the inner and outer layers can be formed by coextrusion. Coextrusion processes are described in, for example, U.S. Patent Application Publication No. US 2002/0165523 A1, published on Nov. 7, 2002, and U.S. patent application Ser. No. 10/351,695, filed on Jan. 27, 2003, and entitled “Multilayer Balloon Member”, both of which are incorporated herein by reference.

Certain of the above-described embodiments include a bumper, typically attached to or integral with the inner member at a position proximal the endoprosthesis. The bumper can reduce the possibility of the endoprosthesis moving proximally as outer member is retracted proximally. In some embodiments, the bumper is formed of a polymeric material, such as a polyether-block co-polyamide polymer (e.g., PEBAX®) or a thermoplastic polyurethane elastomer (e.g., Pellethane™). In certain embodiments, the bumper is made of a metal or an alloy, such as, for example, stainless steel, Nitinol and/or platinum.

Endoprosthesis Construction

In certain embodiments, a self-expanding endoprosthesis (e.g., a stent, stent-graft, or graft) is employed. The self-expanding endoprosthesis can be formed of metals, alloys, polymers, or a combination thereof. Suitable materials include, for example, a stainless steel, polymers, including but not limited to PTFE or PET, and fabrics such as DACRON™. In some embodiments, the endoprosthesis includes a shape-memory material, e.g., a shape memory alloy or a shape memory polymer. Shape memory alloys include nickel-titanium alloy (e.g., Flexinol®, manufactured by Dynalloy, Inc. of Costa Mesa, Calif.), nitinol (e.g., 55% nickel, 45% titanium), silver-cadmium (Ag—Cd), gold-cadmium (Au—Cd), gold-copper-zinc (Au—Cu—Zn), copper-aluminum-nickel (Cu—Al—Ni), copper-gold-zinc (Cu—Au—Zn), copper-zinc/(Cu—Zn), copper-zinc-aluminum (Cu—Zn—Al), copper-zinc-tin (Cu—Zn—Sn), copper-zinc-xenon (Cu—Zn—Xe), iron beryllium (Fe3Be), iron platinum (Fe3Pt), indium-thallium (In—Tl), iron-manganese (Fe—Mn), nickel-titanium-vanadium (Ni—Ti—V), iron-nickel-titanium-cobalt (Fe—Ni—Ti—Co) and copper-tin (Cu—Sn). Other suitable shape memory alloys are described in Schetsky, L. McDonald, “Shape Memory Alloys”, Encyclopedia of Chemical Technology (3rd ed.), John Wiley & Sons, 1982, vol. 20. pp. 726-736, incorporated herein by reference. Shape memory polymers include natural materials, synthetic materials, or a mixture of natural and synthetic materials. In some embodiments, the polymeric material includes a natural polymer, e.g., zein, casein, gelatin, gluten, serum albumin, collagen, polysaccharides, polyhyaluronic acid, poly(3-hydroxyalkanoate)s, alginate, dextran, cellulose, collagen or mixtures of these polymers. In some embodiments, the polymeric material includes a synthetic polymer, e.g., chemical derivatives of collagen, chemical derivatives of cellulose, polyphosphazenes, poly(vinyl alcohols), polyamides, polyacrylates, polyalkylenes, polyacrylamides, polyalkylene glycols, polyalkylene oxides, polyalkylene terephthalates, polyvinyl ethers, polyvinyl esters, polyvinyl halides, polyvinylpyrrolidone, polyesters, degradable polymers, polyester amides, polyanhydrides, polycarbonates, polyorthoesters, polylactides, polyglycolides, polysiloxanes, polyurethanes, cellulose derivatives or mixtures of these polymers. In some embodiments, polymeric material includes mixtures of natural and synthetic polymers. In some embodiments, the polymeric material is cross-linked. The polymer can be, for example, selected from polynorbornene, polycaprolactone, polyenes, nylons, polycyclooctene (PCO), blends of PCO and styrene-butadiene rubber, polyvinyl acetate/polyvinylidinefluoride (PVAc/PVDF), blends of PVAc/PVDF/polymethylmethacrylate (PMMA), polyurethanes, styrene-butadiene copolymers, polyethylene, trans-isoprene, blends of polycaprolactone and n-butylacrylate, and blends thereof.

In certain embodiments, the endoprosthesis is no more than about 60 mm (e.g., no more than about 55 mm, no more than about 50 mm, no more than about 45 mm, no more than about 40, no more than about 35 mm, or no more than about 30 mm) long and/or no less than about 20 mm (e.g., no less than about 25 mm, no less than about 30 mm, no less than about 35 mm, no less than about 40 mm, no less than about 45 mm, or no less than about 50 mm) long.

While certain embodiments have been described, others are possible.

For example, in certain embodiments, the coefficient of friction of the inner surface of the outer member, the outer surface of the inner member, the outer and/or inner surface of the wedge, and/or the outer surface of the endoprosthesis can vary. For example, the system can be configured such that the friction increases as the outer member is retracted. The increase can be, for example, linear, providing a steady increase in friction as the outer member is retracted to make up for the decreasing amount of surface-to surface contact between the outer member and the endoprosthesis and corresponding loss of resistance to distal displacement of the endoprosthesis.

As another example, in some embodiments, the system can include one or more markers (e.g., radiopaque markers). The markers can be used, for example, to help locate the endoprosthesis before the outer member is retracted. In certain embodiments, the markers are carried by the inner member and/or the outer member, the endoprosthesis (e.g., at a distal point on the endoprosthesis and/or at a proximal point on the endoprosthesis), or a combination of these. In some embodiments, the bumper is formed of radiopaque material.

As another example, while systems including a self-expanding stent have been described, other types of implantable medical endoprostheses can be used in the systems. For example, the implantable medical endoprosthesis can be a balloon-expandable implantable medical endoprostheses (e.g., a balloon-expandable stent). In such systems, an inner member would typically include an expandable balloon in a region around which the implantable medical endoprostheses is exposed during delivery. Additional examples of implantable medical endoprostheses include stent-grafts and filters (e.g., arterial filters, venus filters).

As a further example, while embodiments have been described in which the inner and/or outer members have circular transverse cross-sections, in some embodiments the inner and/or outer members can have a noncircular transverse cross-section (e.g., an ovoid transverse cross-section or a polygonal transverse cross-section).

As another example, in some embodiments, the coating on the inner surface of the outer member, the outer surface of the inner member, and/or the outer surface of the endoprosthesis is created by a pultrusion process.

Other embodiments are in the claims.

What is claimed is:

1. A system, comprising:
an inner member;
an outer member disposed about the inner member so that an implantable endoprosthesis can be disposed between the inner and outer members; and
a friction-enhancing device configured to reduce distal movement of the inner member when the outer member is moved proximally.

2. The system of claim 1 further comprising an implantable endoprosthesis disposed between the inner and outer members, wherein the friction-enhancing device comprises an etched inner surface of a distal region of the outer member, an etched outer surface of the implantable endoprosthesis, or both.

3. The system of claim 1 further comprising an implantable endoprosthesis disposed between the inner and outer members, wherein the friction-enhancing device comprises a wedge disposed between the inner member and the outer member at a location proximal the implantable endoprosthesis and having an outer surface in contact with an inner surface of the outer member.

4. The system of claim 3, wherein the wedge has an etched outer surface.

5. A system, comprising:
an inner member having a distal region; and
an outer member having a distal region that surrounds the distal region of the inner member so that an implantable endoprosthesis can be disposed between the distal region of the inner member and the distal region of the outer member;
wherein the distal region of the outer member has inner surface with an etched region.

6. The system of claim 5, the outer member further comprising a proximal region having an interior surface, wherein the inner surface of the proximal region of the outer member has a coefficient of friction at least about 10% less than a coefficient of friction of the etched region of the inner surface of the distal region of the outer member.

7. The system of claim 5, wherein the distal region of the outer member is from about 20 mm to about 60 mm long.

8. A system, comprising:
an inner member having a distal region; and
an outer member having a proximal region and a distal region, the distal region surrounding the distal region of the inner member so that an implantable endoprosthesis can be disposed between the distal region of the inner member and the distal region of the outer member;
wherein the proximal and distal regions of the outer member each have an inner surface having a coefficient of friction, the coefficient of friction of the inner surface of the proximal region of the outer member being different than the coefficient of friction of the inner surface of the distal region of the outer member.

9. The system of claim 8, wherein the inner surface of the distal region of the outer member comprises a material selected from the group consisting of PFA, nylon, PEEK, thermoplastic urethane, and polyethylene.

10. The system of claim 8, wherein the coefficient of friction of the inner surface of the distal region of the outer member is higher than the coefficient of friction of the inner surface of the proximal region of the outer member.

11. A self-expanding implantable endoprosthesis having an etched outer surface.

12. A self-expanding implantable endoprosthesis having an endoprosthesis body with an outer surface and having a coating on the outer surface of the endoprosthesis body, wherein the coating comprises a material having a coefficient of friction higher than the coefficient of friction of the outer surface of the endoprosthesis body.

13. A system comprising:
an inner member;
an outer member; and
a self-expanding implantable endoprosthesis disposed between the inner and outer members;
wherein the endoprosthesis has an etched outer surface.

14. A system comprising:
an inner member having a distal region; and
an outer member having a distal region that surrounds the distal region of the inner member;
an implantable endoprosthesis disposed between the distal region of the inner member and the distal region of the outer member, the implantable endoprosthesis having an outer surface having a coefficient of friction;
a wedge attached to the inner member, the wedge having an outer surface in contact with an inner surface of the outer member, the outer surface of the wedge having a coefficient of friction higher than the coefficient of friction of the outer surface of the endoprosthesis.

15. The system of claim 14, wherein the outer surface of the wedge has a coefficient of friction of at least about 0.25.

16. The system of claim 14, wherein the outer surface of the wedge is etched.

17. The system of claim 14, wherein the outer surface of the wedge comprises polyether-type thermoplastic polyurethane.

18. The system of claim 14, wherein the wedge is attached to the inner member.

19. The system of claim 14, wherein the wedge is configured to allow fluid to pass from a proximal side of the wedge to a distal side of the wedge.

20. The system of claim 14, wherein the wedge comprises a flat wire coil.

21. A system comprising:
an inner member having a distal region;
an outer member having a distal region that surrounds the distal region of the inner member;
an implantable endoprosthesis disposed between the distal region of the inner member and the distal region of the outer member, the implantable endoprosthesis having an outer surface having a coefficient of friction; and
a wedge attached to the outer member, the wedge having an inner surface in contact with an outer surface of the inner member, the inner surface of the wedge having a coefficient of friction higher than the coefficient of friction of the outer surface of the endoprosthesis.

22. The system of claim 21, wherein the inner surface of the wedge is etched.

23. The system of claim 21, wherein the inner surface of the wedge has a tacky surface.

24. The system of claim 21, wherein the wedge is configured to allow fluid to pass from a proximal side of the wedge to a distal side of the wedge.

25. The system of claim 21, wherein the wedge comprises a flat wire coil.

26. A system, comprising:
an inner member having a distal region;
an outer member having a distal region that surrounds the distal region of the inner member so that an implantable endoprosthesis can be disposed between the distal region of the inner member and the distal region of the outer member; and
means for reducing distal movement of the implantable endoprosthesis when the outer member is retracted.

27. The system of claim 26, wherein the means for preventing distal movement of the implantable endoprosthesis is selected from the group consisting of a roughened inner surface of the distal region of the outer member, a coating on the inner surface of the distal region of the outer member, a roughened outer surface of the endoprosthesis, a coating on a portion of the outer surface of the endoprosthesis, a wedge attached to the inner member at a location proximal to the endoprosthesis and having an outer surface with a higher coefficient of friction than an outer surface of the endoprosthesis, and combinations thereof.

28. The system of claim 26, wherein the means for prohibiting distal movement of the implantable endoprosthesis comprises a coating on an inner surface of the distal region of the outer member, the coating having a higher coefficient of friction than an inner surface of a proximal region of the outer member.

29. The system of claim 26, wherein the means comprises a coating, having a higher coefficient of friction than the outer surface of the endoprosthesis, on a portion of the outer surface of the endoprosthesis.
